(19) United States
(12) Patent Application Publication
(10) Pub. No.: US 2005/0231688 A1
(43) Pub. Date: Oct. 20, 2005

(54) Retinal screening using a night vision device

(76) Inventors: Peter W. J. Jones, Belmont, MA (US); Dennis W. Purcell, Medford, MA (US)

Correspondence Address: FISH & NEAVE IP GROUP;ROPES & GRAY LLP, ONE INTERNATIONAL PLACE, BOSTON, MA, 02110-2624, US

(21) Appl. No.: 11/096,409

(22) Filed: Apr. 1, 2005

Related U.S. Application Data
(60) Provisional application No. 60/558,894, filed on Apr. 1, 2004.

(51) Int. Cl. A61B 3/10; A61B 3/00

(52) U.S. Cl. 351/246; 351/221

(57) ABSTRACT

Systems and Methods for viewing an interior portion of an eye in a substantially dark ambient environment using an image intensifier, a color night vision device, or generally a low light sensitive imager capable of outputting an image, including color image.

### CROSS REFERENCE TO RELATED APPLICATIONS

This application claims priority to provisional application Ser. No. 60/558894, filed Apr. 1, 2004, entitled Method of Using Night Vision Goggles for Eye Examination, the contents of which are incorporated herein.

### BACKGROUND OF THE INVENTION

Ophthalmic exams are useful for early detection of vision problems often associated with one or more abnormalities in an eye. The American Academy of Ophthalmology and the American Association for Pediatric Ophthalmology and Strabismus recommend timely screening for early detection and treatment of eye and vision problems, even in very young children. Timely detection of vision and eye problems provides one of the best opportunities for effective, inexpensive treatment and/or prevention of permanent vision loss or degeneration.

In particular, the American Academy of Ophthalmology and the American Association for Pediatric Ophthalmology and Strabismus recommend that a comprehensive vision exam be performed on children—as early as in their infancy—who have a family history of, and possibly at risk to develop, any of the following conditions, among others: retinopathy of prematurity, retinoblastoma, glaucoma, cataracts, retinal dystrophy or degeneration, or any systemic disease associated with the eye and vision.

Another group for whom an early detection of vision or eye problems is important includes diabetics. According to the American Academy of Ophthalmology, approximately 16 million Americans have diabetes. Five million Americans may lose their vision because they do not know that they have the disease. And each year, between 12,000 and 24,000 Americans lose their sight because of diabetes. Eye diseases associated with diabetes include, without limitation, diabetic retinopathy, cataracts, and glaucoma. Among these, the leading cause of new cases of blindness among working-age Americans is diabetic retinopathy. Diabetic retinopathy is a potentially vision-threatening condition in which the blood vessels of the retina are damaged. The damaged vessels may leak, bleed, or scar, and cause retinal detachment, hemorrhaging, macular edema, or a combination of these. It is beneficial, therefore, to have a comprehensive eye exam, including a retinal exam, conducted regularly, especially for the categories of people described above, as well as other population groups.

Currently, at least a portion of a thorough eye exam typically includes dilating a patient's pupil for studying the retina. Generally, a medicated eye drop is applied to the patient's eye to cause the pupil to dilate. This method has several drawbacks. For example, the time it takes for the eye drops to take effect is non-negligible, causing a patient to remain longer than necessary in an eye care facility. This can be a problem, not only for the patient whose time is valuable, but also for an eye care professional who has to deal with such delays in multiple patients during the course of a day, and the resulting reduction in patient throughput. Additionally, the dilated pupil ordinarily takes several hours to return to a normal aperture, causing discomfort to the patient, sometimes to the point of pain; in some cases, the medically-induced pupil dilation precipitates acute glaucoma or mydriasis, endangering the patient's vision, possibly leading to blindness. Vision under normal lighting conditions becomes strenuous for the patient having a dilated pupil, making otherwise ordinary activities, such as driving, particularly hazardous. Moreover, close-up vision can be impaired for several hours following the examination, until the pupil returns to its normal state. The slow return of a dilated retina to its normal aperture also implies that the retinal portion of the exam typically is conducted after the portion studying the eye's refraction properties, thereby forcing a procedural chronology on the eye care professional and the patient.

Attempts to bypass the use of medication to dilate the pupils have been made, typically by illuminating the eye with an infrared beam of light, in a dark surrounding, and measuring the reflected infrared light by an infrared-sensitive instrument, such as an ordinary night vision device. A principle behind these attempts has been that in darkness the pupil dilates naturally and sufficiently fast. An infrared beam does not generally cause the pupil to contract. The examination may be performed at or near darkness, by measuring the infrared light reflected from the retina. After the examination, a visible light is turned on, and the pupil returns to a natural aperture reasonably quickly thereafter.

However, such methods also suffer from serious drawbacks. For example, infrared beams are known to cause retinal damage, even when the beam appears to be dim. In particular, a beam in the near-infrared region of the electromagnetic spectrum (corresponding to wavelengths of about 0.7 microns to about 1.3 microns) may damage the retina or the choroid of the eye, and even cause blindness, possibly without even first creating a sensation of bright light or pain by way of a warning to the subject. A second disadvantage of using infrared light for observing the retina is that infrared light does not allow discrimination of red from some other colors. Therefore, distinguishing blood vessels of the retina from their surroundings, and from each other, becomes very difficult, if not impossible.

Other applications exist that require a careful examination of the retina. In a biometric application, for example, retinal scanning is used to identify a subject. Each retina is, for all practical purposes, unique to the subject to whom the retina belongs.

Retinal scanning involves analyzing blood vessels at the back of the eye. Scanning typically employs a low-intensity light source and an optical coupler that can discern vessel patterns at a particular level of accuracy. The subject whose retina is being scanned typically has an eye close to the device and focuses on a designated point, looking through a small opening in the device at a typically green, or substantially green, light. Ordinarily, identification takes about 10 to 15 seconds to complete, with the eye fixated on the green light in the meantime. However, current hardware used in retinal scanning is prohibitively expensive, and the lack of adaptability of the hardware to new technology has meant that only high-end security applications, for example, secret portions of military installations or other security-sensitive facilities, such as power plants, etc., have been suitable, or economically viable, candidates for the use of a retinal scanner to control access by subjects.

There is therefore a need for an improved eye examination method and device that requires no medication-based dilation of the pupil, nor any of the common discomforts of a medication-based pupil dilation. Additionally, there is also a need for an improved method and/or device to observe the inner cavity of the eye, including the retina.

### SUMMARY OF THE INVENTION

To address the shortcomings of the prior art, the invention, in one embodiment, includes a method of viewing an interior portion of an eye in a substantially dark ambient environment using an image intensifier, a color night vision device, or generally a low-light sensitive imager capable of outputting a color image. In one aspect, the low-light sensitive imager, the image intensifier, or the color night vision system includes an image intensifier tube having microchannels to guide photons. In another aspect, the color night vision system, the image intensifier, or the low-light-sensitive imager includes a CMOS chip, a CCD (charge-coupled device), or other light-sensitive electronic circuitry.

The method includes capturing a visual representation of the interior portion of the eye through a pupil of the eye, and illuminating the eye with a visible light source having a brightness adjusted to allow the color night vision device to capture the visual representation while the pupil maintains a sufficiently dilated aperture. In one aspect, capturing the visual representation includes processing light reflected from the interior portion of the eye by an image intensification tube. Optionally, the color night vision system may be operably connected to a display monitor for viewing of the visual representation. According to one practice, the color night vision device is connected to a computer or other data processing platform. The computer, in a particular embodiment, may include, or be in communication with, a database, a data storage medium, or both.

Optionally, the computer according to the systems and methods described herein includes a software executing on the computer, in response to the visual representation of the retina. In one embodiment, the software includes an algorithm to enhance at least one feature of the visual representation, or to otherwise modify the visual representation. Alternatively, or additionally, the software executing on the computer may issue a control to the color night vision device in response to a feature of the visual representation.

If the system according to the invention includes a database for storing retinal or other data associated with one or more eyes, then the software may include one or more algorithms to compare one or more features of the visual representation captured by the color night vision device and the data stored in the database. In one particular aspect, comparing the one or more features of the visual representation with the data stored in the database includes seeking to determine a match between the one or more features and the data stored in the database.

In one embodiment, the visual representation captured by the color night vision device is used to track a movement of the eye. In an alternative embodiment, the visual representation is used to discern a pattern of retinal vessels or other features, including, without limitation, status of an optic nerve, integrity of the retina, health of the macula, and other features associated with the health of the eye or a biometric of the subject to whom, or to which, the eye belongs.

In one embodiment, capturing the visual representation of the retina includes modifying at least a portion of an electromagnetic spectrum reaching the color night vision device. For example, and without limitation, the portion being modified may include an infrared portion of the electromagnetic spectrum. In one aspect, the infrared portion is suppressed by the inventive systems and methods described herein, before light reaches the color night vision device.

In one embodiment, the color night vision system is coupled, perhaps through the computer, to a treatment device for the eye. The computer, in response to the visual representation captured by the color night vision device, issues at least one command to the treatment device to control an operation of the device. The treatment device, in one practice, includes a laser treatment device used, for example, to treat abnormalities associated with glaucoma. However, it is clear to those of skill in the art that other treatment devices exist that can be used with the systems and methods described herein.

Other objects of the invention will, in part, be obvious, and, in part, be shown from the following description of the systems and methods shown herein.

### BRIEF DESCRIPTION OF THE DRAWINGS

The foregoing and other objects and advantages of the invention will be appreciated more fully from the following further description thereof, with reference to the accompanying drawings, wherein;

FIG. 1A depicts a section of a mammalian eye;

FIG. 1B depicts a view of a retina, including some detail of interest in a retinal scan, as seen from the front of the eye;

FIG. 2A depicts a bright light illuminating the eye, causing a pupil to contract, thereby narrowing the viewing angle;

FIG. 2B depicts a bright light illuminating the eye, wherein the pupil of the eye has been dilated by an application of medicated eye drops, thereby providing a wide viewing angle;

FIG. 2C depicts an exemplary use of an ophthalmoscope having a two-way mirror, to examine the eye.

FIG. 3 depicts the pupil naturally dilated in a dark room, the pupil illuminated by a light source sufficiently dim so as to not cause contraction of the pupil, thereby providing a wide angle of view for a color night vision system to capture a visual representation of the retina;

FIG. 4 depicts an image intensifier tube used by a color night vision device;

FIG. 5 depicts the setup of FIG. 3, including interconnections between the color night vision system and the light source, and optional data storage and/or processing systems.

### DESCRIPTION OF THE ILLUSTRATED EMBODIMENTS

To provide an overall understanding of the invention, certain illustrative embodiments will now be described, including a system that provides for viewing of a mammalian retina in substantially dark surroundings, employing a color night vision system and a light source illuminating the retina with a visible light sufficiently dim so as to not cause dilation of the pupil. However, it will be understood by one of ordinary skill in the art that the systems and methods described herein can be adapted and modified for other suitable applications and that such other additions and modifications will not depart from the scope hereof.

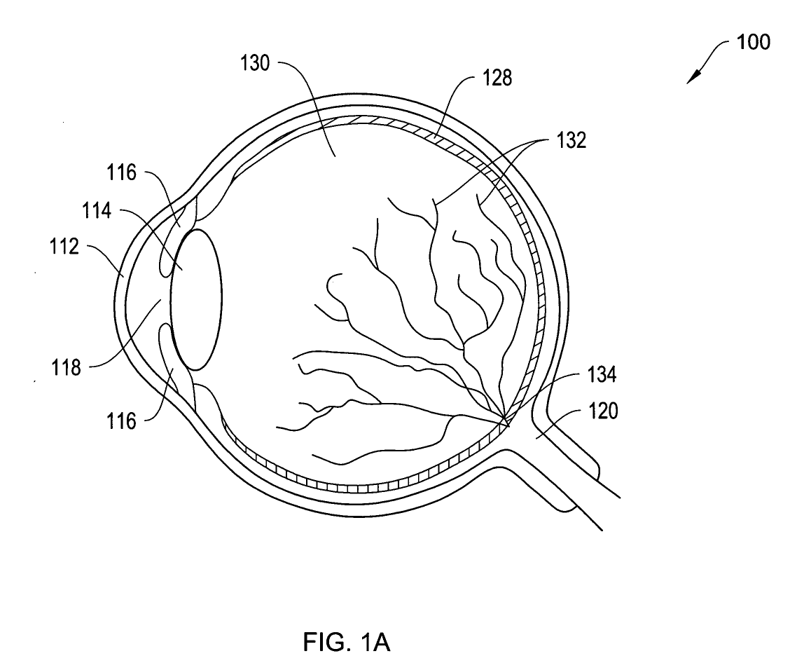

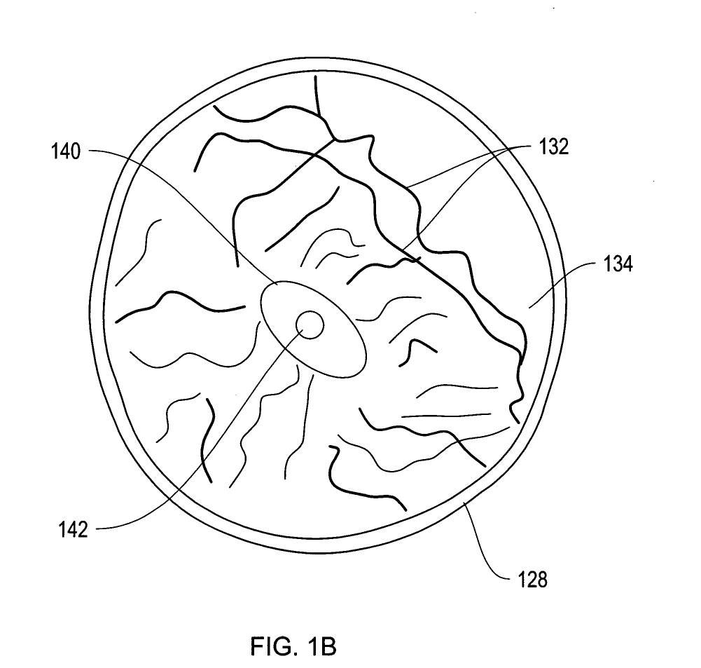

Turning to FIG. 1A, a familiar view of a section of a mammalian eye 100 is depicted. By way of a review, the eye 100 includes a cornea 112, a lens 114, and an iris 116 disposed between the cornea and the lens. A pupil 118 is defined by the opening of the iris. A retina 128 lines a back portion of the interior of the eye 100. The retina has rod cells (not shown), responsible for low-light vision, and cone cells (not shown), responsible for color vision, that in turn send signals to a brain (not shown) through an optic nerve 120 disposed at the posterior of the eye, carrying electrical impulses to the brain. Where retinal blood vessels 132 and the optic nerve 120 meet is the optic disc 134. A thick, transparent substance 130, called the vitreous, and composed mainly of water, occupies about ⅔ of the volume of the eye 100.

FIG. 1B depicts a view of the retina 128 as seen from the front of the eye 100. FIG. 1B includes some of the salient features of interest to a retinal screening. For example, in the retinal screening there may be interest in assessing a state of the retinal vessels 132; retinal vessels may be arteries or veins. In one application, the pattern of the retinal vessels 132 is of interest. For example, in a retinal scan for identification, the pattern of the retinal vessels 132 is sought, because this pattern is substantially unique to the individual mammal to which the retina 128 belongs.

In another practice, the general health of the retinal vessels is of interest. For example, an eye care professional may be interested in ascertaining that the vessels 132 do not suffer from any abnormality, such as, without limitation, a leak or rupture; if there is a retinal vessel abnormality, the eye care professional may conduct a retinal screening to discern the extent of the abnormality of the retina 128.

Near the center of the retina 128, in the back of the eye 100, is the macula 140. Substantially at the center of the macula 140 is the fovea 142. In some applications, the state, such as the health, of the macula and the fovea are of interest. For example, an eye care professional may be interested in performing a retinal screening to discover whether the patient suffers from a type of macular degeneration or a macular hole. In yet another application, the health of the optic disc 134 is of interest.

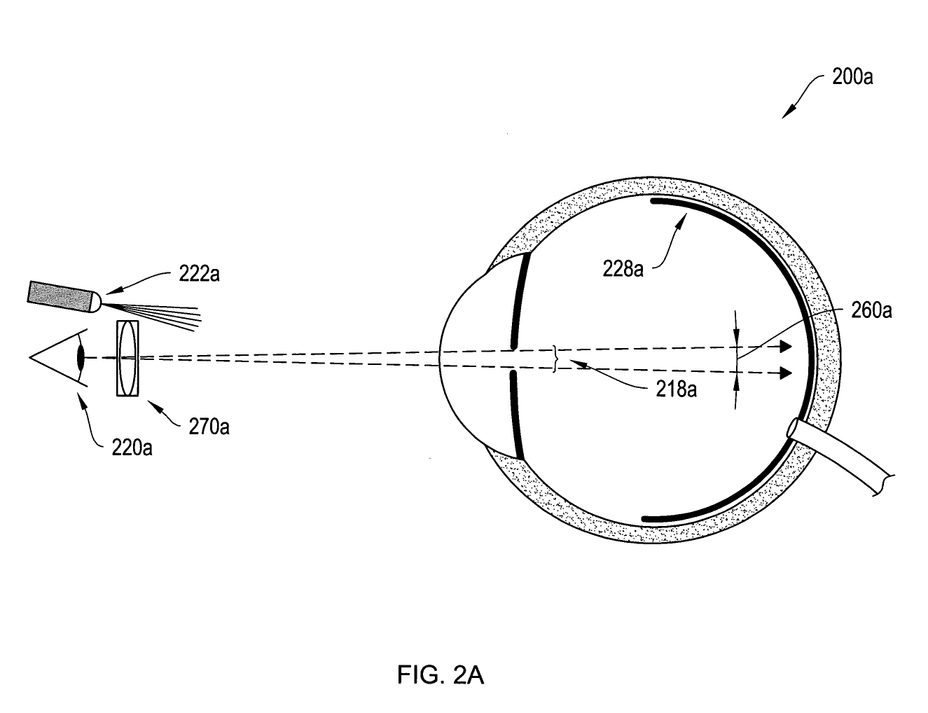

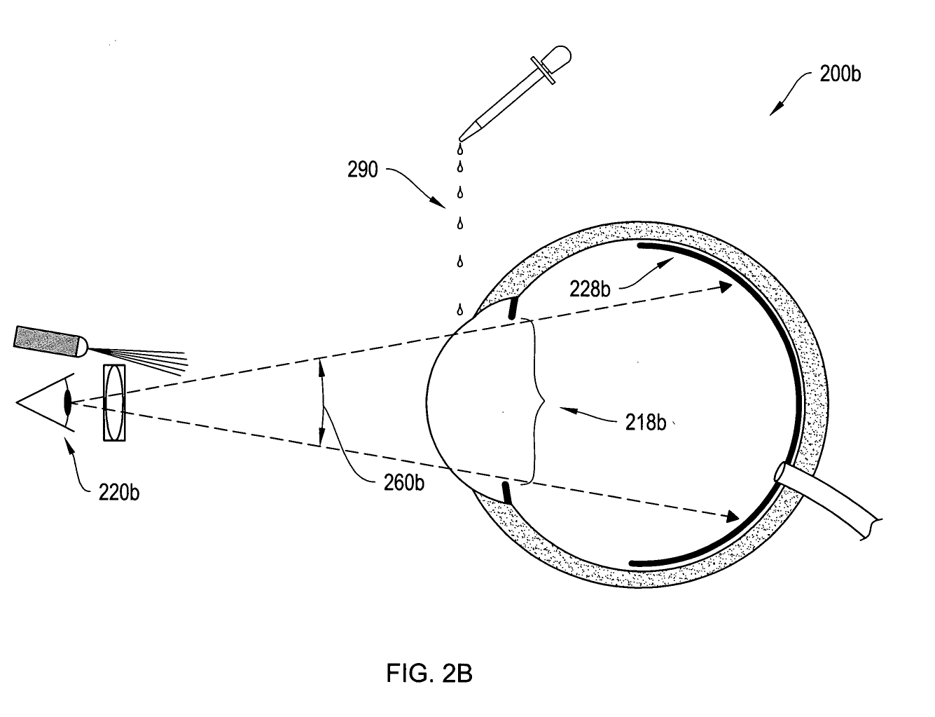

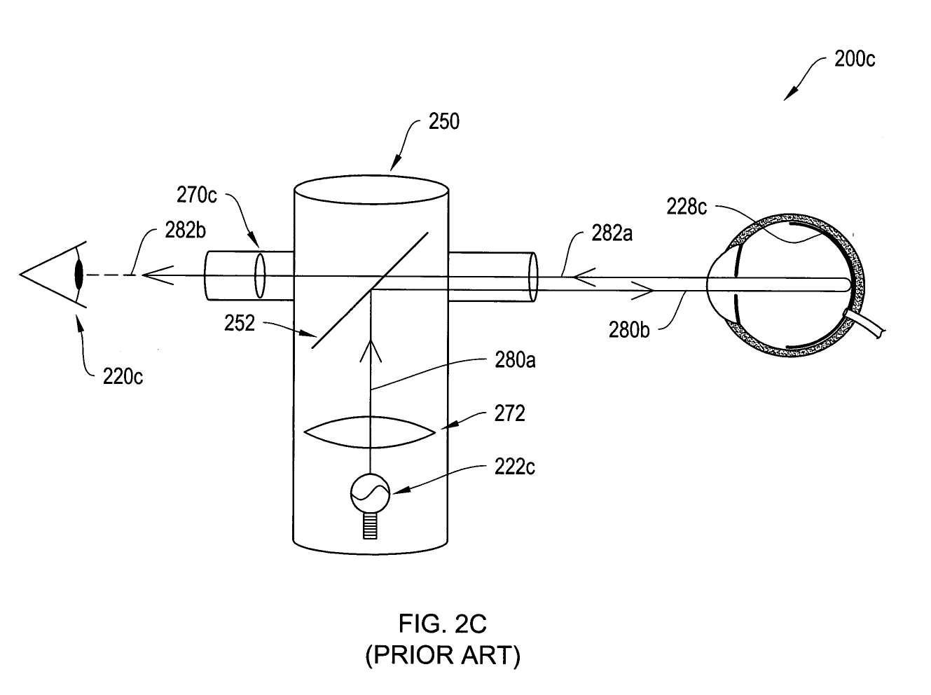

Turning to FIG. 2A, a problem associated with illuminating the eye 200a by employing a bright light source 222a is depicted. In the context of an eye examination, for example, an observer, such as, without limitation, an ophthalmologist or other eye care professional 220a typically uses the bright light source 222a and an auxiliary lens 270a (for example, a focusing lens or another configuration of optical elements/devices) to observe an interior portion of the eye 200a. The bright light illuminated by the source 222a, however, typically causes the pupil to contract to a small opening 218a. This hamper's the observer's view of the interior of the eye, narrowing the angle of view 260a, thereby preventing most of the retina 228a from being observed.

FIG. 2B shows how, to remedy this problem—that is, to obtain a wider angle of view—medicated drops 290 are typically applied to the eye 200b, causing the pupil 218b to dilate to a large opening. This medically-induced dilation of the pupil is unnatural, because the pupil remains dilated regardless of how bright the ambient lighting is. The dilation of the pupil 218b allows the observer 220b to have a wider angle of view 260b of the retina 228b. As mentioned earlier, the need to use dilation drops 290 increases the time required for an exam, thereby decreasing patient throughput in an eye care facility. The drops 290 take time to manifest their effect on the pupil 218b. Moreover, subsequent to the exam, the dilated pupil 218b typically takes several hours to return to a normal state. The dilated pupil also causes irritation to the eye, especially when exposed to bright light. Reading is made difficult, walking outside without very dark glasses impossible, and driving dangerous.

FIG. 2C depicts an alternative setup for conducting an eye exam using an ophthalmoscope. In FIG. 2C, an observer, such as an eye care professional 220c, is shown looking through an ophthalmoscope 250 toward retina 228c of a patient's eye 200c. The ophthalmoscope 250 includes a two-way mirror 252, commonly known as a half-silvered mirror; a property of the two-way mirror 252 is that it reflects a first portion and transmits a second portion of the light incident upon the mirror. In one embodiment, the two-way mirror 252 reflects approximately 50% of a light incident upon it, and transmits the remaining approximately 50% of the light. Two exemplary rays of light indicating the operation of the ophthalmoscope 250 are shown in FIG. 2C. A light ray 280a emitted by a light source 222c is partially reflected from the two-way mirror 252. The reflected portion 280b travels toward the retina 228c of the eye 200c. Another portion (not shown) of the light ray 280a is transmitted through the two-way mirror 252. Light reflecting from the retina 228c returns as the exemplary ray 282a along the direction of the dashed line shown in FIG. 2C. A portion (not shown) of the light ray 282a is reflected by the two-way mirror 252. However, another portion, namely, 282b, is transmitted through the two-way mirror 252 along the direction shown by the arrow of the dashed line depicted by 282b, toward the observer, usually an eye care professional 220c. The ophthalmoscope may optionally include additional optical elements, such as, without limitation, a first focusing lens 270c (analogous to the focusing lenses 270a and 270b of FIGS. 2A and 2B) and a second focusing lens 272.

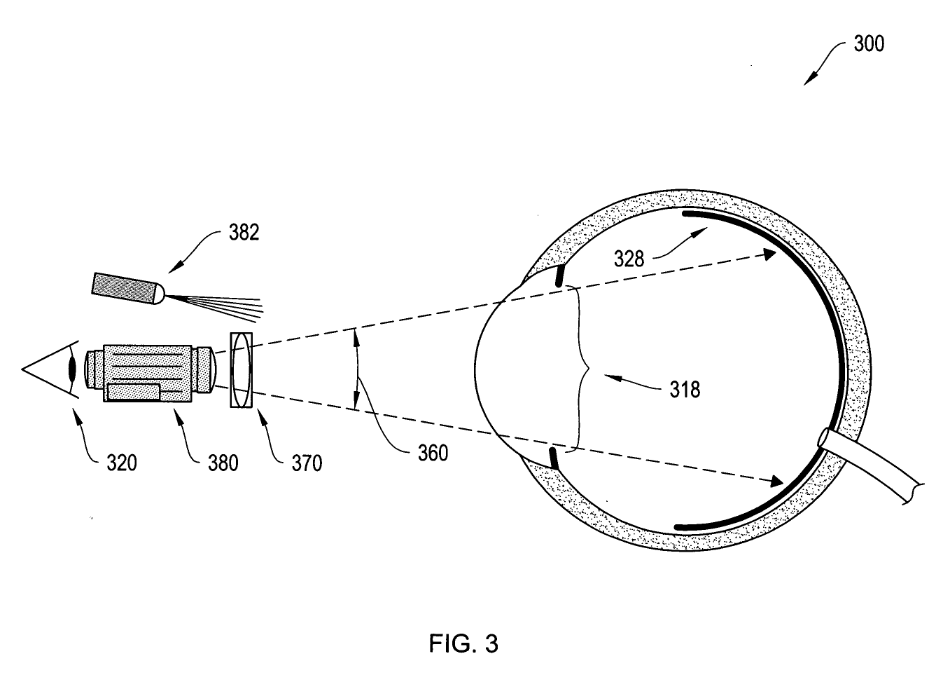

Turning to FIG. 3, an embodiment of the invention is depicted to remedy the problems associated with medically-induced dilation of the eyes, as described above in relation to FIG. 2B. In particular, FIG. 3 shows an embodiment of the invention used by an observer 320, typically an eye care professional such as an ophthalmologist or an optometrist, viewing the eye 300 in a substantially dark ambient environment. As is well known, the human eye adjusts to the dark ambient environment as the pupil 318 dilates naturally, in response to the darkness, providing a wide angle of view 360 for observation of the retina 328. According to the embodiment of the invention depicted by FIG. 3, a color night vision system 380 is employed in the dark environment to view a portion of the interior of the eye 300, in particular the retina 328. Use of a color night vision system such as the system 380 is desirable, at least in part because eye care professionals are trained in, and are accustomed to, viewing color images of the retina 328. Furthermore, a monochrome image of the retina 328 can be difficult to analyze. The principles underlying the operation of color night vision systems and methods of producing color night vision imagery are known in the art. For example, and without limitation, the color night vision system 380 may be one that is made and/or used, according to a subset of the methods and systems disclosed in a subset of the following patent and non-patent published references: U.S. Pat. No. 6,614,606, issued on 2 Sep. 2003; U.S. Pat. No. 5,214,503, issued on 25 May 1993; “Fusion of Multi-Sensor Imagery for Night Vision: Color Visualization, Target Learning and Search”, by D. A. Fay et al., Proceedings of Fusion 2000: 3^{rd} International Conference on Information Fusion, Paris, France, 2000; “Color Night Vision: Opponent Processing in the Fusion of Visible an IR Imagery”, Waxman et al., Neural Networks, vol. 10, no. 1, pp. 1-6, 1997; “Color Night Vision for Navigation and Surveillance”, Das, S. et al., In J. Sutton and S. C. Kak (Eds), Proceedings of the Fifth Joint Conference on Information Sciences, Atlantic City, N.J., 28 Feb. 2000.

To provide adequate lighting, a light source 382 is employed that has a brightness sufficiently high to provide a meaningful observation of the interior of the eye 300, and sufficiently low so as to not cause a contraction of the pupil 318. As the color night vision system can operate under low-light conditions, the light source 382 typically illuminates the eye 300 with substantially dim lighting. In one embodiment, the light source 382 may have variable brightness. For example, and without limitation, the light source 382 may be initially bright enough to maintain the pupil 318 in a contracted state. Then, the operator 320, a computer (not shown), or the color night vision system 380 can dim the brightness of the light source 382, controlling the pupil's dilation according to a desired dilation speed and/or pattern. Once the pupil 318 is sufficiently dilated and/or stabilized in the dilated state, the operator 320 can perform a study of the retina 328. Typically, the light source 382 emits light that is at least partially achromatic and/or non-coherent.

The color night vision system 380 may optionally be coupled to an optical system to filter one or more portions of the electromagnetic spectrum reaching the system 380. For example, the optical system 370 may include a narrow-band blocking filter to accentuate retinal surface detail information. Alternatively, or additionally, the optical system 370 may include an infrared filter to modify at least a portion of the infrared light reaching the color night vision system 380. In an embodiment, it is desirable to eliminate or reduce a near-infrared portion of the electromagnetic spectrum, corresponding to a subset of a wavelength range of about 0.7 micron to about 1.3 microns.

Alternatively, in an embodiment, the light source 382 is configured so that it emits visible light primarily in the blue-green portion of the electromagnetic spectrum, and its emission in the range of the color red is significantly diminished. This is at least partially because radiant energy in the near-infrared portion of the electromagnetic spectrum is strongly absorbed by water. Water is more transparent to the blue-green portion of the electromagnetic spectrum. Therefore, the range of frequencies from blue to green provide good penetration through the vitreous, which, as mentioned earlier in relation to FIG. 1, is composed mainly of water. As a result, discriminating features of the eye 300 becomes easier. In an alternative embodiment, the light source 382 emits primarily white light, but is coupled to an optical system (not shown) that primarily allows the blue to green portion of the electromagnetic spectrum to pass through to the eye 300.

Alternatively, or additionally, it may be desirable to eliminate or reduce a portion of the electromagnetic spectrum corresponding to a subset of a wavelength range of about 1.3 microns to about 3 microns. In another aspect, it may be desirable to eliminate or reduce a thermal-infrared portion of the electromagnetic spectrum corresponding to a subset of a wavelength range of about 3 microns to about 30 microns or longer.

Elimination or reduction of at least a portion of the infrared range of electromagnetic waves can be useful in, for example, eliminating or reducing some of the known artifacts associated with infrared imaging, such as correct color rendition; it was mentioned earlier that infrared imaging does not provide an acceptable rendition of the retina's blood vessel pattern, for example. Alternatively, or additionally, an optical system (not shown) may be coupled to the light source 382 to modify features of the light emitted by the light source 382. For example, the infrared filtering discussed above may be implemented, in one embodiment, by the optical system coupled to the light source 382.

The color night vision system 380 may include color night vision goggles, a color night vision monocular, color night vision binoculars, a device, such as a medical device in which a color night vision system is incorporated, or any of the variety of color night vision devices, or low-light sensitive color imagers, known in the art. Typically, though not exclusively, the color night vision system 380 obtains visible imagery by using one or more late generation image intensifier tubes (usually three)—such as Generation III intensifier tubes—optically coupled to a conventional charge coupled device (CCD). Modern color night vision systems are capable of producing remarkably realistic color renditions of scenes in substantially dark environments.

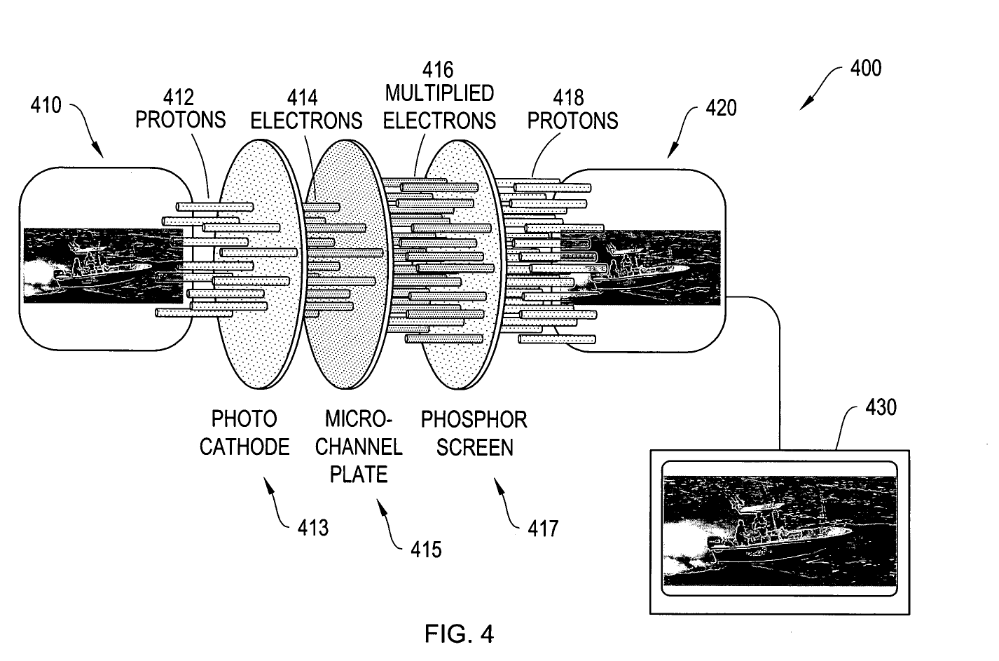

An image intensifier tube 400 is depicted by FIG. 4. The image intensifier tube converts photons of light to electrons, multiplies the electrons manifold, and converts the multiplied electrons back to photons, amplifying the number and/or energy of the photons in the process; the amplified photons are employed to create an enhanced visual representation 420 of an original, actual, typically low-light scene 410. A travel path through the image intensifier tube 400 is typically as follows. Ambient light photons 412 from the low-light scene 410 reach an objective lens (not shown), which focuses an image of the scene 410 on a photocathode 413 of the image intensifier tube 400. The photocathode 413 is used to convert the photons 412 to electrons 414. As the electrons 414 travel through the tube 400, they pass through a microchannel plate (MCP) 415. Typically, the MCP is held in a vacuum, and is a small, glass disc, bearing millions of microscopic holes, called microchannels; the microchannels generally are longer than they are wide, sometimes by a factor of around 45.

A metal electrode (not shown) is attached to each side of the MCP. When the electrons 414 hit the first electrode of the MCP, they are accelerated through the microchannels by a large voltage burst between the electrode pair. As the electrons 414 travel through the microchannels, they collide with the interior linings of the microchannels, exciting thousands of other electrons, causing the release of those thousands of electrons, via a process called cascaded secondary emission. The released electrons themselves collide with the interior walls of the microchannels, releasing yet more electrons, in a cascading phenomenon. Eventually, the multiplied electrons 416 exit the MCP. Near the terminus of the intensifier tube, the multiplied electrons 416 hit a phosphor screen 417, which is used to convert the electrons into photons 418, larger in energy and number than the photons 412. The photons 418 typically are viewed in the form of an image after they pass through an ocular lens (not shown) substantially near the terminus of the tube 400. The result is the much stronger visual representation 420 of the original scene 410. Optionally, the image 420 may be viewed on an electronic display, such as a monitor 430, and it may be stored in a digital form for subsequent analysis or viewing.

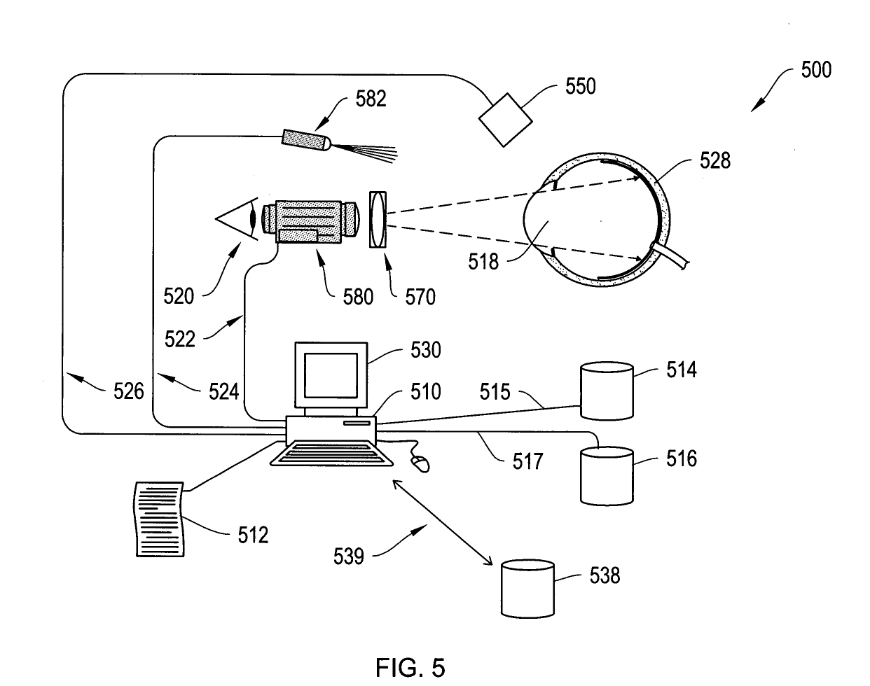

Turning to FIG. 5, an embodiment of the invention is shown having many optional features. A color night vision system 580 is used to observe the retina 528, through the pupil aperture 518. Optionally, the color night vision system 580 is coupled to an optical system 570 to filter a portion of the electromagnetic spectrum reaching the system 580. As mentioned earlier, the optical system 570 may include an infrared blocking feature to reduce the amount of infrared light reaching the system 580. Alternatively, or additionally, the optical system 570 may include a feature enhancing filter, to emphasize some features of the visual representation of the retina 528. The filtering of the portion of the electromagnetic spectrum discussed above, and features of the light source 582 can be similar to those described in relation to FIG. 3 earlier.

In one practice, the color night vision system 580 is coupled to a computer 510 having an optional monitor 530 on which a visual representation of the retina 528 can be displayed and viewed. The computer 510 is optionally equipped with software 512 that can perform one or more actions in response to the visual representation of the retina 528. For example, and without limitation, the software 512 may include a pattern recognition algorithm used to identify one or more features of the retina 528; extracting a pattern of the retinal vessels or features of the optic disc (not shown), or identifying an individual to whom the retina 528 belongs are sample tasks that can be performed by the software 512 executing on the computer 510.

Alternatively, the software 512 may be used to determine the dilation of the pupil 518, based on the visual representation captured by the color night vision device 580. Upon determination of the extent of the pupil's dilation 518, the software 512 executing on the computer 510 may issue a set of one or more control commands to the color night vision system 580, via communication link 522, to adjust parameters of the system 580. The parameters may include, for example and without limitation, a lens aperture, sensitivity, exposure time, image capture rate (for a video sequence), and other setting parameters associated with the system 580. Optionally, link 522 may be used by the color night vision system 580 to convey one or more features of the environment that the system is operating in, to the computer 510 or the software 512. In one aspect, the software 512, in the course of executing on the computer 510 to perform one or more tasks, requests data from the color night vision system 580, that is in turn conveyed by the system 580 via link 522.

Optionally, the software 512 executing on the computer 510 may issue a set of one or more commands to the light source 582, adjusting, for example, the brightness of the light source 582 or controlling a duration of the illumination provided by the light source 582. In one embodiment, the light source emits a bright light, but pulsed over a short enough duration as to provide capturing of an acceptable image of the retina 528 before the pupil 518 contracts in response to the bright flash of light.

In yet another embodiment, the software 512 executing on the computer 510 includes one or more image enhancement algorithms used to alter the visual representation of the retina 528 captured by the color night vision system 580. Alternatively, the software 512 may include a tracking algorithm to track the movement of the eye, the tracking being in response to a video sequence of images of the retina 528, the pupil 518, or both, captured by the color night vision device 580.

Optionally, the computer 510 may be connected, or it may include, a storage medium 514 for storing data captured by the color night vision system 580. The visual information captured by the color night vision system 580 may, in one embodiment, be compared with data residing on a database 516 containing analogous data. For example, in the context of retinal scanning for the purpose of identification, the visual representation of the subject's retina 528 may be compared with a database of retinal information stored in the database 516, to determine if there is a match. In this embodiment, the software 512 may provide a matching algorithm for this purpose. Alternatively, the database 516 may include software to perform the matching. The database 516 and the storage 514 are communicatively linked, in one or more embodiments, to the computer 510 by links 517 and 515, respectively. In one embodiment, the computer 510 is communicatively linked to a data processor 538, via link 539. The remote device may be, for example and without limitation, another computer, a remote client device, a server, or a combination of these. In general, the computer 510 or the data processor 538 may be a personal computer, a workstation, a personal digital assistant, a notebook computer, a tablet PC, or any other computing platform.

The computer 510 may, in one embodiment, be communicatively coupled to a treatment device 550, via link 526. The treatment device 550 may be, for example and without limitation, a laser treatment device for correcting an abnormality of the eye, such as glaucoma, or burst or leaking retinal vessels, etc.

Any combination of the links 515, 517, 522, 524, 526, and 539 may be hardwired or wireless links. The computer 510, the storage 514, the database 516, the color night vision system 580, and light 582, the treatment device 550, and the data processor 538 may be interconnected through a local area network, a wide area network, a wireless network, a fiber optic network, or a combination of these and other networking architectures known in the art.

The computer 510, by communicating with the remote data processor 538, may engage in telemedicinal interactions, according to one embodiment. The data processor 538 may be a remote computing platform capable of processing the data, displaying the data for a remote user, or otherwise conveying the data captured from the retina 528 to a remote location for viewing, analysis, storage, etc. Communication of data between the computer 510 and the data processor 538 may be through an e-mail client, a web-based client, ftp protocol, or other transfer modalities known in the art.

Although the embodiments shown in FIGS. 2A, 2B, 3, and 5 do not depict the use of a two-way mirror, such as the two-way mirror 252 of FIG. 2C, it is understood by one of ordinary skill in the art that the half-silvered mirror 252 may be used in one or more aspects of the invention according to one or more of the FIGS. 2A, 2B, 3, and 5.

Many equivalents to the specific embodiments of the invention described herein, and the specific methods and practices associated with the invention, exist. Applicants contemplate and consider within the patentable subject matter of this application, all operable combinations of the illustrative features, elements, systems, devices, and methods described herein for observing, measuring, and recording of information pertaining to the interior of an eye in general, and the retina of the eye in particular, including any and all associated parts, such as, without limitation, blood vessels (arteries and veins) and their pattern; macula; optic nerve; optic disk; fovea; cup; rods; cones; or a combination thereof. Applicants also contemplate and consider within the patentable subject matter of the invention, all applications wherein a method or device is employed to observe the interior of the eye, such as, without limitation, a biometric identification using a retinal scanner. The application of the invention is understood to include any mammal. One area of application, for example, is in tagging and identification of wildlife or domesticated mammals. For example, retinal scans of wild or domesticated animals can be performed for the purpose of identification and/or tagging.

Accordingly, the invention is not to be limited to the embodiments, methods, and practices disclosed herein, but is to be understood from the following claims, which are to be interpreted as broadly as allowed under the law.

What is claimed is:

1. A method of viewing an interior portion of an eye in a substantially dark ambient environment, comprising:
a. using a color night vision device, capturing a visual representation of the interior portion of the eye through a pupil of the eye; and
b. illuminating the eye with a visible light source having a brightness adjusted to allow the color night vision device to capture the visual representation while the pupil maintains a sufficiently dilated aperture.

2. The method of claim 1, wherein capturing the visual representation includes processing light reflected from the interior portion of the eye by an image intensification tube.

3. The method of claim 1, wherein capturing the visual representation includes transferring the representation to a display monitor for viewing.

4. The method of claim 1, including connecting the color night vision device to a computer.

5. The method of claim 4, wherein the computer includes a storage medium for storing the visual representation.

6. The method of claim 4, wherein the computer includes a database for storing data associated with at least one other eye.

7. The method of claim 4, wherein the computer includes at least one software executing on the computer in response to the visual representation.

8. The method of claim 7, wherein the executing includes enhancing at least one feature of the visual representation.

9. The method of claim 7, wherein the executing includes issuing a control to the color night vision device in response to a feature of the visual representation.

10. The method of claim 7, wherein the executing includes issuing a control to the light source in response to the visual representation.

11. The method of claim 6, including comparing one or more features of the visual representation with the data stored in the database.

12. The method of claim 11, including seeking a match between at least one parameter associated with the visual representation and at least one parameter associated with the data stored in the database.

13. The method of claim 1, including tracking a movement of the eye in response to the visual representation.

14. The method of claim 1, including identifying a feature of the eye in response to the visual representation.

15. The method of claim 14, wherein the feature includes a pattern of retinal blood vessels.

16. The method of claim 14, wherein the feature includes the status of an optic nerve associated with the eye.

17. The method of claim 1, wherein capturing the visual representation includes modifying at least a portion of an electromagnetic spectrum reaching the color night vision device.

18. The method of claim 17, wherein the portion of the electromagnetic spectrum includes an infrared portion.

19. The method of claim 18, wherein the modifying includes reducing the infrared portion.

20. The method of claim 1, including treating the eye in response to the visual representation.

21. The method of claim 20, wherein the treating includes employing a laser treatment device.

22. The method of claim 20, wherein the treating includes applying medication to the eye in response to the visual representation.

23. A method of viewing an interior portion of an eye in a substantially dark ambient environment, comprising:
a. using an image intensifier, capturing a visual representation of the interior portion of the eye through a pupil of the eye; and
b. illuminating the eye with a visible light source having a brightness adjusted to allow the image intensifier to capture the visual representation while the pupil maintains a sufficiently dilated aperture.

24. The method of claim 23, wherein the image intensifier includes an image intensifier tube.

25. The method of claim 23, wherein the image intensifier is configured to output a color image.

26. A method of viewing an interior portion of an eye in a substantially dark ambient environment, comprising:
a. using a low-light sensitive device, capturing a visual representation of the interior portion of the eye through a pupil of the eye; and
b. illuminating the eye with a visible light source having a brightness adjusted to allow the low-light sensitive device to capture the visual representation while the pupil maintains a sufficiently dilated aperture.

27. The method of claim 26, wherein the low-light sensitive device includes an image intensifier tube.

28. The method of claim 26, wherein the low-light sensitive device includes a CMOS electronic chip.

29. The method of claim 26, wherein the low-light sensitive device includes a CCD.

30. The method of claim 26, wherein the low-light sensitive device is configured to output a color image.
